(19) United States
(12) Patent Application Publication
(10) Pub. No.: US 2004/0137428 A1
(43) Pub. Date: Jul. 15, 2004

(54) Method and device for identifying pathogenic agents

(76) Inventor: Philip S. Low, West Lafayette, IN (US)

Correspondence Address: BARNES & THORNBURG, 11 South Meridian Street, Indianapolis, IN, 46204, US

(21) Appl. No.: 10/640,359

(22) Filed: Aug. 13, 2003

Related U.S. Application Data
(60) Provisional application No. 60/402,979, filed on Aug. 13, 2002.

(51) Int. Cl. C12Q 1/70; G01N 33/53; G01N 33/569; G01N 33/554

(52) U.S. Cl. 435/5; 435/7.22; 435/7.31; 435/7.32

(57) ABSTRACT

The present invention provides a method and a device for the identification of a pathogenic agent in a sample. The method comprises the steps of contacting the pathogenic agent in the sample with a plurality of ligands selected from the group consisting of an oligosaccharide, a siderophore, a ferrisiderophore, a vitamin, analogs or derivatives thereof, and combinations thereof, wherein the plurality of ligands are non-diffusably bound to a solid support in a spatially defined manner, detecting binding of the pathogenic agent to at least one of the ligands, wherein the pathogenic agent binds specifically to the at least one ligand to generate a spatially defined binding pattern, and identifying the pathogenic agent based on the spatially defined binding pattern. The device comprises a solid support, and a plurality of ligands bound to a surface of the solid support wherein the plurality of ligands comprises an oligosaccharide and an iron-chelating agent, or analogs or derivatives thereof, and wherein the plurality of ligands is bound to the surface of the solid support in a spatially defined manner.

### CROSS REFERENCE TO RELATED APPLICATION

This application claims priority under 35 U.S.C. § 119(e) to U.S. Provisional Application No. 60/402,979 filed Aug. 13, 2002.

### FIELD OF THE INVENTION

The present invention relates to a method and a device for use in identifying pathogenic agents. More particularly, pathogen binding oligosaccharides, siderophores, or ferrisiderophore complexes, vitamins, or other essential ligands, or their pathogen binding analogs or derivatives, or combinations thereof, are linked to a solid support for use in identifying pathogenic agents.

### BACKGROUND AND SUMMARY OF THE INVENTION

Pathogenic agents are inherently difficult to identify. They rapidly undergo mutation to alter characteristics typically used for their identification, such as cell surface characteristics, metabolic responses, and behavior in culture. Moreover, many diagnostic techniques in current use for pathogen identification lack the speed, sensitivity, and specificity essential for such time-critical assays. Therefore, a need exists for diagnostic assays that can be used to identify definitively and rapidly pathogenic agents in biological or nonbiological samples by targeting pathogen characteristics that cannot be changed without a decrease in or a loss of virulence.

Pathogenic agents have unique classes of cell surface receptors that are necessary for maximal pathogenicity in the host. These unique classes of receptors include 1.) receptors on the surface of pathogenic agents that bind to oligosaccharides found on host cells providing a means for attachment of the pathogenic agents to host cells, 2.) receptors for binding iron-chelating compounds secreted by pathogenic agents under conditions where the level of iron is low in the extracellular environment, and 3.) receptors for vitamins. Each of these unique receptors are necessary for maximal pathogenicity of pathogenic agents in the host, and, in some cases, are essential for survival and virulence of pathogenic agents. Thus, mutations in these receptors that inhibit ligand binding are selected against, making the capacity of these receptors to bind their respective ligands a trait that is useful for identification of pathogenic agents.

Oligosaccharide binding receptors on the surface of pathogenic agents are important to pathogenicity and survival of these organisms in the host because binding to host cell surface oligosaccharides is a major mechanism of attachment to host cells. Attachment of pathogenic agents to host cells is an important factor in infectivity and virulence. For example, attachment to host cells can lead to internalization of the pathogenic agent and to compromise of signaling pathways in the host. The capacity to impinge on host intracellular signaling is often an important step in pathogenesis. Thus, many pathogenic agents have receptors on their surfaces for host cell oligosaccharides, and the capacity to bind to these oligosaccharides with high affinity and high specificity is necessary for maximal pathogenicity and, in some cases, survival of the pathogenic agent in the host.

Acquisition of iron is required for the pathogenicity and survival of pathogenic agents in a host because iron is an essential nutrient for these organisms. In the extracellular compartment pathogens are confronted with an environment in which the level of iron is extremely low because iron is sequestered by the host proteins transferrin and lactoferrin. Thus, the ability to compete with lactoferrin and transferrin for iron is essential for the pathogenicity, and, potentially for the survival of pathogenic agents in the host. Accordingly, in an iron-deficient environment most pathogens secrete low molecular weight iron-chelating compounds known as siderophores to facilitate iron acquisition. Siderophores are high affinity iron-chelating compounds that are capable of removing transferrin- or lactoferrin-bound iron to form ferrisiderophore complexes. Pathogenic agents have high affinity, high specificity receptors for siderophores and ferrisiderophore complexes on their surfaces. Upon iron chelation, ferrisiderophore complexes have been observed to displace iron-free siderphores bound to the surface of pathogenic agents, and the ferrisiderophore complexes are internalized into the organism where the iron is released. Therefore, pathogenic agents have specific binding sites for siderophores and ferrisiderophore complexes on their membrane surfaces, and the capacity to bind ferrisiderophore complexes is necessary for infectivity and virulence of pathogenic agents in the host. Accordingly, mutations that significantly alter siderophore- and ferrisiderophore-receptor interactions should be selected against, or, if they occur, may prove lethal.

Pathogenic agents, such as Escherichia coli, have receptors on their surfaces for vitamins. The capacity to bind to vitamins with high affinity and high specificity is necessary for the virulence of these pathogenic agents in the host so receptors for vitamins constitute another trait of pathogenic agents that is useful for their identification.

Biochip arrays have become an increasingly important tool in the biotechnology industry and related fields for the identification and quantitation of unknown analytes. A biochip is a device that consists of a recognition molecule, such as a ligand, linked to a solid support, and a transducer based on an integrated circuit microchip. The interaction of an analyte, the biological species to be analyzed, with the recognition molecule produces a signal that can be measured by the transducer (e.g., an electrical signal, a visual signal, etc.). Biochips commonly employ such interactions as 1.) antibody/antigen interactions, 2.) nucleic acid hybridizations, 3.) enzyme/substrate interactions, 4.) cellular interactions, and 5.) interactions using synthetic biomimetic bioreceptors. Conventional signals detectable by a transducer are 1.) optical signals, 2.) electrochemical signals, and 3.) signals generated by changes in mass. Typically, biochips consist of a plurality of recognition molecules (e.g., ligands) deposited onto a solid support in the form of an array or a pattern, and the recognition molecules can be individually monitored for use in analyzing multiple analytes. Such biochip arrays can be used in diagnostic applications to identify and quantify an unknown pathogenic agent.

The present invention is directed to an array comprising ligands including oligosaccharides, siderophores, ferrisiderophore complexes, vitamins, or other essential ligands, or analogs or derivatives thereof, or combinations thereof, linked to a solid support for the identification of unknown pathogens in biological or nonbiological samples. The invention takes advantage of 1.) the capacity of pathogenic agents to bind with high affinity and specificity to host cell oligosaccharides, 2.) the capacity of pathogenic agents to bind with high affinity and specificity to siderophores and ferrisiderophore complexes, 3.) the capacity of pathogenic agents to bind with high affinity and specificity to vitamins, and 4.) the requirement for binding to oligosaccharides, siderophores, ferrisiderophore complexes, and vitamins for virulence, and in some cases survival, of pathogenic agents in the host resulting in selection against mutations that inhibit binding to these ligands. Accordingly, binding to these ligands is a trait of pathogens useful for their identification.

In one embodiment, a method of identifying a pathogenic agent in a sample is provided. The method comprises the steps of contacting the sample with a plurality of ligands selected from the group consisting of an oligosaccharide, a siderophore, a ferrisiderophore, and a vitamin, or pathogen binding analogs or derivatives thereof, or a combination thereof, wherein the plurality of ligands are non-diffusably bound to a solid support in a spatially defined manner, detecting binding of the pathogenic agent to at least one of the ligands, wherein the pathogenic agent binds specifically to the at least one ligand to generate a spatially defined binding pattern, and identifying the pathogenic agent based on the spatially defined binding pattern.

In another embodiment, a method is provided of making a device for use in identifying a pathogenic agent in a sample, the device comprising a plurality of ligands bound to a solid support, the plurality of ligands comprising an oligosaccharide, a pathogen-binding analog or derivative thereof, and an iron-chelating agent, or a pathogen binding analog or derivative thereof. The method comprises the steps of making and/or purifying the plurality of ligands, contacting the plurality of ligands with the solid support; and attaching the plurality of ligands to the solid support in a spatially defined manner.

In yet another embodiment, a device for use in identifying a pathogenic agent in a biological sample is provided. The device comprises a solid support and a plurality of ligands bound to a surface of the solid support wherein the plurality of ligands comprises an oligosaccharide, or a pathogen binding analog or derivative thereof, and an iron-chelating agent, or a pathogen binding analog or derivative thereof, and wherein the plurality of ligands is bound to the surface of the solid support in a spatially defined manner.

### DETAILED DESCRIPTION OF THE INVENTION

The present invention can be used to identify pathogenic agents in a variety of biological or nonbiological samples such as blood, urine, a stool sample, a sputum sample, a gavage, a tissue sample, semen, cerebrospinal fluid, pus, amniotic fluid, tears, saliva, a lung aspirate, a vaginal or urethral discharge, a plant extract, a water sample, an air sample, a soil sample, and a foodstuff. Biological samples also include samples collected with swabs from the skin or throat. The method and device of the present invention can also be used to identify pathogenic agents harbored by infected cells where the infected cells can be disrupted to release the pathogenic agents.

The present invention can be used to analyze the presence or absence of a pathogenic agent in a sample or the amount of a pathogenic agent in a sample.

The method and device of the present invention have both human and veterinary applications. Thus, the animals harboring the pathogenic agents can be humans or, in the case of veterinary applications, can be laboratory, agricultural, domestic, or wild animals. The present invention can be used for animals including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales. The samples containing the pathogenic agents can also be of plant origin.

The invention is applicable to pathogenic agents that cause a variety of infectious diseases. For example, the present invention is applicable to such pathogenic agents as bacteria, fungi, including yeasts, viruses, mycoplasma, and parasites. Pathogenic agents that can be identified using the method and device of the present invention are any art-recognized pathogenic agents that cause disease in an animal or a plant, including such organisms as bacteria that are gram-negative or gram-positive cocci or bacilli. For example, Proteus species, Klebsiella species, Providencia species, Yersinia species, Erwinia species, Enterobacter species, Salmonella species, Serratia species, Aerobacter species, Escherichia species, Pseudomonas species, Shigella species, Vibrio species, Aeromonas species, Campylobacter species, Streptococcus species, Staphylococcus species, Lactobacillus species, Micrococcus species, Moraxella species, Bacillus species, Bordetella species, Enterococcus species, Propionibacterium species, Streptomyces species, Clostridium species, Corynebacterium species, Eberthella species, Micrococcus species, Mycobacterium species, Neisseria species, Haemophilus species, Bacteroides species, Listeria species, Erysipelothrix species, Acinetobacter species, Brucella species, Pasteurella species, Vibrio species, Flavobacterium species, Fusobacterium species, Streptobacillus species, Calymmatobacterium species, Legionella species, Treponema species, Borrelia species, Leptospira species, Actinomyces species, Nocardia species, Rickettsia species, and any other bacterial species that causes disease in an animal or plant can be identified with the method and device of the present invention.

Viruses, such as DNA and RNA viruses, can also be identified in accordance with the invention. Such viruses include, but are not limited to, DNA viruses such as papilloma viruses, parvoviruses, adenoviruses, herpesviruses and vaccinia viruses, and RNA viruses, such as arenaviruses, coronaviruses, rhinoviruses, respiratory syncytial viruses, influenza viruses, picornaviruses, paramyxoviruses, reoviruses, retroviruses, and rhabdoviruses.

The present invention is also applicable to any fungi, including yeasts, mycoplasma species, parasites, or other pathogenic agents that cause disease in animals or plants. Examples of fungi that can be identified with the method and device of the present invention include fungi that grow as molds or are yeastlike, including, for example, fungi that cause diseases such as ringworm, histoplasmosis, blastomycosis, aspergillosis, cryptococcosis, sporotrichosis, coccidioidomycosis, paracoccidio-idomycosis, mucormycosis, chromoblastomycosis, dermatophytosis, protothecosis, fusariosis, pityriasis, mycetoma, paracoccidioidomycosis, phaeohyphomycosis, pseudallescheriasis, sporotrichosis, trichosporosis, pneumocystis infection, and candidiasis.

The present invention can also be utilized to identify parasites including, but not limited to, tapeworms, such as Taenia, Hymenolepsis, Diphyllobothrium, and Echinococcus species, flukes, such as Fasciolopsis, Heterophyes, Metagonimus, Clonorchis, Fasciola, Paragonimus, and Schitosoma species, roundworms, such as Enterobius, Trichuris, Ascaris, Ancylostoma, Necator, Strongyloides, Trichinella, Wuchereria, Brugia, Loa Onchocerca, and Dracunculus species, ameba, such as Naegleria and Acanthamoeba species, and protozoans, such as Plasmodium, Trypanosoma, Leishmania, Toxoplasma, Entamoeba, Giardia, Isospora, Cryptosporidium, and Enterocytozoon species. To identify such parasites in accordance with the present invention, the membranes of these organisms, or other components, can be isolated and solubilized or extracted for application in a fluidized form to the device of the present invention (e.g., a biochip).

The oligosaccharides for attachment to the solid support in accordance with the invention can be any oligosaccharides, or analogs or derivatives thereof, displayed on the surface of host cells that are involved in attachment of pathogenic agents to the host cells. These oligosaccharides can be identified by methods such as those described in Scharfinan et al., Glycobiol. 9:757-764 (1999), Lingwood, Biopolymers 2:695-700 (1998), and Heikkila et al., J. Infect. Disease 176:704-712 (1997).

A plurality of oligosaccharides (i.e., ligands) is typically attached to the solid support and the plurality of oligosaccharides attached to the solid support can constitute a mixture of the specific oligosaccharide(s) to which each of many different pathogenic agents bind. Accordingly, the oligosaccharides included in the mixture depend on the oligosaccharide binding specificities of the different pathogenic agents to be identified. The pathogenic agents to be identified can bind with high affinity and specificity to one or more of the oligosaccharides attached to the solid support, and the oligosaccharides to which a particular pathogenic agent binds may be different from other pathogenic agents or there may be overlap in the oligosaccharides to which different pathogenic agents bind. The oligosaccharides are attached to the solid support in a spatially defined manner so that a pathogenic agent can be identified based on its spatially defined binding pattern. Thus, in accordance with the invention, a single solid support with attached ligands can be used for the identification of many different pathogenic agents.

The oligosaccharides, or analogs or derivatives thereof, can be made by any art-recognized procedure such as the procedures described in Preparative Carbohydrate Chemistry, S. Hanessian, Ed., 1997, Marcel Dekker, New York, Basel, Hong Kong, and Monosaccharides, Their Chemistry and Their Roles in Natural Products, P. Collins and R. Ferrier, Eds., 1995, John Wiley & Sons, New York, Brisbane, Toronto, Singapore. Any oligosaccharides implicated in host cell attachment can be used including, but not limited to, sialyl lactose, Lewis glycoconjugates (i.e., Lewis blood group oligosaccharides), sulfatides, gangliotriaosyl, gangliotetraosyl ceramide, sulfogangliotetraosyl ceramide, heparan sulfate, lactosyl ceramide, polyglyocsyl ceramide, asialo-ganglioside GM₂, globoside, lacto-N-neotetraose, and sialylated derivatives thereof, and the like. Of particular interest for use in accordance with the invention are multivalent oligosaccharides, or analogs or derivatives thereof.

The siderophores for use in accordance with the invention can be any siderophores or ferrisiderophore complexes, or analogs or derivatives thereof, to which pathogenic agents bind with high affinity and specificity. A plurality of siderophores and/or ferrisiderophore complexes (i.e., ligands) is typically attached to the solid support and the plurality of siderophores and/or ferrisiderophore complexes attached to the solid support can constitute a mixture of the specific siderophores and/or ferrisiderophore complexes to which each of many different pathogenic agents bind. Accordingly, the siderophores and/or ferrisiderophore complexes included in the mixture depend on the siderophores and/or ferrisiderophore complex binding specificities of the different pathogenic agents to be identified.

The pathogenic agents to be identified can bind with high affinity and specificity to one or more of the siderophores and/or ferrisiderophore complexes attached to the solid support, and the siderophores and/or ferrisiderophore complexes to which a particular pathogenic agent binds may be different from other pathogenic agents or there may be overlap in the siderophores and/or ferrisiderophore complexes to which different pathogenic agents bind. The siderophores and/or ferrisiderophore complexes are attached to the solid support in a spatially defined manner so that a pathogenic agent can be identified based on its spatially defined binding pattern to the solid support. The siderophores and/or ferrisiderophore complexes can be attached to the solid support in combination with oligosaccharides, and, in this case, the pathogenic agent is identified based on its spatially defined binding pattern to siderophores and/or ferrisiderophore complexes and to oligosaccharides. Siderophores are secreted by pathogenic agents and can be purified from the culture medium of the pathogens according to art-recognized procedures such as the procedure described in Barnes et al., Biometals 12(1):83-7 (1999), Iijima et al., J. Antibiot. 52(1):20-4 (1999), and Ratledge et al., Microbiology 142(8):2207-12 (1996).

Vitamins for attachment to a solid support can be chosen depending on the vitamin binding specificities of pathogenic agents, and vitamins can be attached to a solid support for detection of pathogenic agents in a manner similar to that described above for siderophores and oligosaccharides. Vitamins that can be utilized in accordance with the invention include niacin, pantothenic acid, folic acid, riboflavin, thiamine, biotin, vitamin B₁₂, vitamins A, D, E and K, other related vitamin molecules, analogs and derivatives thereof, and combinations thereof.

Other essential ligands can also be attached to the solid support for use in identification of pathogenic agents, either alone or in combination with the above-described ligands. In accordance with the invention, “essential ligands” means a ligand to which a pathogenic agent binds wherein ligand binding is important for pathogencity of the pathogenic agent in the host.

The solid support for use in accordance with the invention can be any material that can be modified to contain discrete sites appropriate for the attachment of the oligosaccharides and/or siderophores and/or ferrisiderophore complexes and/or vitamins and/or other essential ligands for use in accordance with the invention. As will be appreciated by those skilled in the art, the number of possible solid supports is large, and includes, but is not limited to, glass and modified or functionalized glass, plastics (including acrylics, polystyrene and copolymers of styrene and other materials, polypropylene, polyethylene, polybutylene, polyurethanes, polycarbonate, etc.), Langmuir Blodgett film, polysaccharides, nylon or nitrocellulose, (poly)tetrafluroethylene, (poly)vinylidendifluoride, resins, germanium, silica or silica-based materials including silicon and modified silicon, carbon, metals, such as gold and silver, and the like. In general, the solid supports do not appreciably fluoresce.

Generally, the solid support is planar, such as in the form of a sheet, a pad, a slice, a film, a plate, a slide, or the like, although other configurations of the solid support can be used. For example, the oligosaccharides and/or siderophores and/or ferrisiderophore complexes and/or vitamins and/or other essential ligands can be attached on the inside surface of a tube for flow-through sample analysis, or the solid support can flexible, such as in the form of a flexible foam, or can be in the form of particles, strands, precipitates, gels, spheres, containers, capillaries, and the like. The solid support can also contain raised or depressed regions on which the oligosaccharides and/or siderophores and/or ferrisiderophore complexes and/or vitamins and/or other essential ligands are attached.

The surface of the solid support should comprise a material capable of having functional groups such as amino, carboxyl, thiol, oxo, olefin, or hydroxyl groups attached to its surface. Typically, these groups are used to covalently attach optional crosslinking agents which are typically covalently linked to oligosaccharides and/or siderophores and/or ferrisiderophore complexes and/or vitamins and/or other essential ligands so that the subsequent binding of the pathogenic agents occurs in solution without interference from the solid support surface. The nature of the crosslinking agents is not critical; they can be any divalent functional moiety. Typical crosslinking groups include ethylene glycol oligomer, diamines, and amino acids. Any suitable technique useful for attaching an oligosaccharides and/or a siderophore and/or a ferrisiderophore complex and/or a vitamin and/or other essential ligands to a solid support is contemplated in accordance with this invention. An example of such a device is a biochip, and methods for making biochips are well-known in the art.

For example, the oligosaccharide, siderophore, ferrisiderophore complex, vitamin, and/or other essential ligand, or analogs or derivatives thereof, can be non-diffusably bound (i.e., attached) to a solid support through the above-described crosslinking groups or directly to functional groups at the surface of the solid support or by utilizing any other art-recognized method of attaching compounds to a solid support. These direct and indirect methods can include covalent, ionic, or hydrogen bonding. The oligosaccharide, siderophore, ferrisiderophore complex, vitamin, and/or other essential ligand is typically attached to the solid support by covalent bonding through the formation of amide, ester or imino bonds between acid, aldehyde, hydroxy, amino, or hydrazo groups. The method of attachment, whether by direct or indirect means (i.e., a crosslinking group), should not prevent the binding of the oligosaccharide, siderophore, ferrisiderophore, vitamin, other essential ligand, or an analog or derivative thereof, to the receptor on the surface of the pathogenic agent. Furthermore, binding of iron to the ferrisiderophore should not be prevented.

The attachment of the oligosaccharide and/or siderophore and/or ferrisiderophore complex and/or vitamin and/or other essential ligand to the solid support in a non-diffusable and spatially defined manner can be achieved by any suitable method including those methods known in the art. In one embodiment, one or more oligosaccharides and/or siderophores and/or ferrisiderophore complexes and/or vitamins and/or other essential ligands are attached to the solid support by photolithography using a photoreactive protecting group on a coupling agent. Such a technique is described in U.S. Pat. No. 5,412,087. Thiolpropionate having a photochemically removable protecting group is covalently coupled to functional groups on the surface of the solid support. Light of the appropriate wavelength is then used to illuminate predefined regions of the surface, resulting in photodeprotection of the thiol group. A mask is used to ensure that photodeprotection takes place only at the desired sites on the surface of the solid support. Oligosaccharides and/or siderophores and/or ferrisiderophore complexes and/or vitamins and/or other essential ligands containing thiol reactive groups, such as maleimides, are then attached to the deprotected regions. The unbound ligands are then washed away, and the process is repeated at another location with another ligand or mixture of ligands.

A similar method uses a 5′-nitroveratryl protected thymidine linked to an aminated solid support via a linkage to the 3′-hydroxyl group (Fodor et al., Science 251: 767-773 (1991)). Photodeprotection of the thymidine derivative allows a phosphoramidite activated monomer (or oligomer) to react at this site. Other suitable methods use a photoactivatable biotin derivative to spatially localize avidin binding (see U.S. Pat. No. 5,252,743 and WO 91/07807).

In another embodiment, to spatially restrict the binding of oligosaccharides and/or siderophores and/or ferrisiderophore complexes and/or vitamins and/or other essential ligands to particular regions of the solid support, the regions that come into contact with the ligands at each attachment photoactivation step are spatially restricted. This can be done by placing the solid support on a block containing channels through which the ligands are pumped, with each channel giving the ligands access to only a small region of the solid support. In this embodiment, a mask allows for the patterned illumination and consequent photoactivation of several regions of the solid support at the same time. If the area surrounding each photoactivated region is segregated from the neighboring region by the channel, then different ligands can be delivered to the photoactivated regions by pumping the ligands through different channels (see U.S. Pat. Nos. 5,384,261, 5,556,752, and 5,677,195).

In further embodiments, the oligosaccharide and/or siderophore and/or ferrisiderophore complex and/or vitamin and/or other essential ligand array is formed on the solid support by methods known in the art, such as methods which utilize an ink-jet, a spray, a piezo-electric, or a bubble jet protein printer, whereby the ligands are deposited by electro-mechanical dispensers at spatially defined locations (see U.S. Pat. No. 5,658,802). Other methods known in the art for forming spatially defined arrays on a solid support include spotting methods (see U.S. Pat. Nos. 5,288,514, 5,312,233, and 6,024,138), bead-based methods (see U.S. Pat. No. 5,541,061), and pin-based methods (see U.S. Pat. Nos. 5,288,514 and 5,566,572).

A form of template-stamping is also contemplated in accordance with the invention, wherein a template containing an ordered array of oligosaccharide and/or siderophore and/or ferrisiderophore complex and/or vitamin and/or other essential ligands is used to deposit the same ordered array on multiple solid supports.

A variety of conditions for contacting (i.e., binding) the pathogenic agent with the oligosaccharide and/or siderophore and/or ferrisiderophore complex and/or vitamin and/or other essential ligand can be used in the present invention, such as variations in temperature, salt concentration, chaotropic salt concentration, pH, solvent concentration, and the like. In addition, reagents such as salts, buffers, neutral proteins (e.g., albumin), detergents, anionic, cationic, nonionic, or amphoteric surfactants, and protease inhibitors can be included in the solutions added to the device to optimize specific binding of the pathogenic agents to the oligosaccharide and/or siderophore and/or ferrisiderophore complex and/or vitamin and/or other essential ligands, and to minimize nonspecific binding.

The spatially defined pattern of binding of the pathogenic agent to oligosaccharide and/or siderophore and/or ferrisiderophore complex and/or vitamin and/or other essential ligands on the surface of the solid support allows identification of the pathogenic agent in the sample. The spatially defined binding patterns on the device (e.g., a biochip) are typically read by optical means, although other methods known in the art can also be used such as electrochemical and mass-sensitive techniques (see Vo-Dinh et al., Fresenius J. Anal. Chem. 366:540-551 (2000)). Although the present method can be used to analyze one sample at a time, the method can also be conducted in high-throughput format to analyze, for example, a plurality of samples simultaneously. The detection of the binding of the pathogenic agent to oligosaccharide and/or siderophore and/or ferrisiderophore complex and/or vitamin and/or other essential ligands attached to the solid support comprises detection of binding to at least one (i.e., one or more) of the ligands.

As will be appreciated by those skilled in the art, the spatially defined binding patterns can be read directly or can be read indirectly, for example, using a “sandwich assay.” A “sandwich assay” requires binding to the pathogenic agent of labeled oligosaccharides and/or siderophores and/or ferrisiderophore complexes and/or vitamins and/or other essential ligands in a second composition. The second composition is added to the device following the binding of the pathogenic agent to be identified to the ligands attached to the solid support. The spatially defined binding pattern of the oligosaccharides and/or siderophores and/or ferrisiderophore complexes and/or vitamins and/or other essential ligands in the second composition can be detected due to the presence of the label. The labeling system can comprise, for example, a fluorescent compound, a chemiluminescent compound, an enzyme, or can produce a chemical or a radioactive signal. The label provides a means of detecting the spatially defined binding pattern due to specific binding of the labeled oligosaccharides and/or siderophores and/or ferrisiderophore complexes and/or vitamins and/or other essential ligands to the pathogenic agent bound to the ligands attached to the solid support. “Sandwich assay” procedures are described in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697.

What is claimed is:

1. A method of identifying a pathogenic agent in a sample, said method comprising the steps of
contacting the sample with a plurality of ligands selected from the group consisting of an oligosaccharide, a siderophore, a ferrisiderophore, and a vitamin, or pathogen binding analogs or derivatives thereof, or a combination thereof, wherein the plurality of ligands are non-diffusably bound to a solid support in a spatially defined manner;
detecting binding of the pathogenic agent to at least one of the ligands, wherein the pathogenic agent binds specifically to the at least one ligand to generate a spatially defined binding pattern; and
identifying the pathogenic agent based on the spatially defined binding pattern.

2. The method of claim 1 wherein the pathogenic agent is selected from the group consisting of bacteria, fungi, viruses, mycoplasma, and parasites.

3. The method of claim 1 wherein the plurality of ligands are chemically complexed to the solid support through bonding comprising covalent, ionic, or hydrogen bonding.

4. The method of claim 1 wherein the sample is selected from the group consisting of blood, urine, a stool sample, a sputum sample, a gavage, a tissue sample, a plant extract, a water sample, a soil sample, an air sample, and a foodstuff.

5. The method of claim 1 wherein the solid support is a biochip.

6. A method of making a device for use in identifying a pathogenic agent in a sample, the device comprising a plurality of ligands bound to a solid support, the plurality of ligands comprising an oligosaccharide, a pathogen-binding analog or derivative thereof, and an iron-chelating agent, or a pathogen binding analog or derivative thereof, the method comprising the steps of
making and/or purifying the plurality of ligands;
contacting the plurality of ligands with the solid support; and
attaching the plurality of ligands to the solid support in a spatially defined manner.

7. The method of claim 6 wherein the plurality of ligands is attached to the solid support through bonding comprising covalent, ionic, or hydrogen bonding.

8. The method of claim 6 wherein the iron-chelating agent is selected from the group consisting of a siderophore and a ferrisiderophore complex.

9. The method of claim 6 wherein the plurality of ligands further comprises a vitamin or a pathogen binding analog or derivative thereof.

10. A device for use in identifying a pathogenic agent in a biological sample, comprising
a solid support; and
a plurality of ligands bound to a surface of the solid support wherein the plurality of ligands comprises an oligosaccharide, or a pathogen binding analog or derivative thereof, and an iron-chelating agent, or a pathogen binding analog or derivative thereof, and wherein the plurality of ligands is bound to the surface of the solid support in a spatially defined manner.

11. The device of claim 10 wherein the plurality of ligands is attached to the surface of the solid support through bonding comprising covalent, ionic, or hydrogen bonding.

12. The device of claim 10 wherein the solid support is a biochip.

13. The device of claim 10 wherein the iron-chelating agent is selected from the group consisting of a siderophore and a ferrisiderophore complex.

14. The device of claim 10 wherein the plurality of ligands further comprises a vitamin or a pathogen binding analog or derivative thereof.
